(19) United States
(12) Patent Application Publication
(10) Pub. No.: US 2017/0166956 A1
(43) Pub. Date: Jun. 15, 2017

(54) Methods for DNA Preparation for Multiplex High Throughput Targeted Sequencing

(71) Applicant: Shoreline Biome, LLC, Farmington, CT (US)

(72) Inventors: Mark Driscoll, Wallingford, CT (US); Thomas Jarvie, Branford, CT (US)

(21) Appl. No.: 15/372,588

(22) Filed: Dec. 8, 2016

Related U.S. Application Data
(60) Provisional application No. 62/266,072, filed on Dec. 11, 2015.

(51) Int. Cl. C12Q 1/68

(52) U.S. Cl. CPC C12Q 1/6806; C12Q 1/6874; C12Q 1/6883; C12Q 1/689; C12Q 2600/156; C12Q 2600/16; C12Q 2600/158

(57) ABSTRACT

Disclosed are methods for parallel single-step DNA purification starting with multiple crude biological samples for subsequent parallel PCR amplification of target DNA that attaches a unique DNA sequence tag (barcode) allowing all parallel processed samples to be combined into a single high-throughput sequencing run. The methods disclosed herein can be used to prepare and sequence dozens or hundreds of targeted samples as part of a rapid, highly parallel process, after which individual sample sequencing results are separated using the sample-specific tags (barcodes) to obtain results for each sample.

### CROSS-REFERENCE TO RELATED APPLICATIONS

This application claims the benefit under 35 U.S.C. §119(e) to U.S. Provisional Application No. 62/266,072, filed Dec. 11, 2015, the contents of which are incorporated herein by reference in their entirety.

### FIELD OF THE DISCLOSURE

Disclosed are methods for parallel single-step DNA purification starting with multiple crude biological samples for subsequent parallel PCR amplification of target DNA that attaches a unique DNA sequence tag (barcode) allowing all parallel processed samples to be combined into a single high-throughput sequencing run. The methods disclosed herein can be used to prepare and sequence dozens or hundreds of targeted samples as part of a rapid, highly parallel process, after which individual sample sequencing results are separated using the sample-specific tags (barcodes) to obtain results for each sample.

### BACKGROUND OF THE DISCLOSURE

Until recently, the majority of the cost of DNA sequencing applications was driven by the costs associated with DNA sequencing itself, rather than DNA preparation or analysis of the results. Recent advances in the Next Generation DNA Sequencing (NGS) field have resulted in sequencing throughput gains and associated sequencing cost decreases sufficient for dozens or hundreds of targeted samples to be sequenced together (multiplexed samples). An example of targeted samples are amplicons, where only a limited region of the entire genome of an organism is to be sequenced. Amplicon sequencing is a type of targeted sequencing commonly used in diagnostic sequencing tests, where only a portion of the genome is targeted, for example human BRCA 1-2 genes that indicate risk for breast cancer, or specific mutations in viral genomes known to influence drug efficacy. Multiplexing samples decreases sequencing costs in direct relationship to the number of samples per run, so that the major expenditure of time and cost of targeted NGS sequencing for targeted sequencing has shifted to preparation of the dozens to hundreds of individual samples needed to fill a high throughput sequencing run to make it cost effective. Practical methods for preparation of DNA for high throughput targeted sequencing applications should be simple, fast, inexpensive, and scalable from dozens to hundreds of samples. Current best protocols tend to require many steps, are difficult to perform manually or use complex automation, and the protocols yield varying quantities of DNA, requiring additional steps for quantitation and dilution. These limitations make sample preparation for typical NGS sequencing assays expensive, limiting NGS utility for high throughput sequencing of specific DNA targets. Here we describe an adaptation of a DNA amplification protocol as a high throughput sample preparation method, we combine it with the use of multiple barcodes (unique sequence tags) for sample tracking, enabling samples to be mixed in a single high throughput sequencing run. The combination enables a highly streamlined workflow for assaying many samples at once using a targeted, multiplexed Next Generation DNA Sequencing approach.

### SUMMARY OF THE INVENTION

Disclosed herein are methods for high throughput processing of large numbers of samples for sequencing using Next Generation Sequencing platforms, comprising of: (a) amplifying whole genome DNA from biological samples in a high throughput format, wherein the biological samples are selected from crude biological samples or partially-purified biological samples; (b) for each sample, amplifying one or more target DNA sequences from the genomic DNA from step (a), wherein each targeted DNA sequence has a unique DNA barcode corresponding to the identity of the sample, enabling all samples to be pooled into a single NGS DNA sequencing library; (c) conducting high throughput DNA sequencing on the target DNA sequences from step (b); and (d) separating the DNA sequences obtained in step (c) by sample barcode, and using the separated DNA sequences to identify the microbes present in each sample.

In some embodiments, the methods further comprising, prior to step (a), lysing cells in the biological sample so as to release DNA from the cells. Cell lysis may be carried out using reagents selected from the group consisting of: enzymes such as lysozyme or proteinase, a base such as KOH or NaOH, a detergent such as nonyl phenoxypolyethoxylethanol (NP-40; CAS number 9016-45-9), 3-[(3-cholamidopropyl)dimethylammonio]-1-propanesulfonate (CHAPS; CAS number 75621-03-3), C₁₄H₂₂O(C₂H₄O)ₙ (n=9-10) (Triton X-100; CAS number 9002-93-1), or sodium dodecyl sulfate (SDS). NP-40, CHAPS, Triton X-100 and SDS are available from commercial manufacturers including Sigma-Aldrich Co, LLC (St. Louis, Mo.) and ThermoFisher Scientific, Inc. (Waltham, Mass.).

In some embodiments, the high throughput format is selected from the group consisting of: at least six samples, at least twenty-four samples, at least 48 samples, at least ninety-six samples, at least 384 samples or at least 1536 samples.

In some embodiments, the biological sample is selected from the group consisting of: feces, cell lysate, tissue, blood, tumor, tongue, tooth or, buccal swab, phlegm, mucous, wound swab, skin swab, vaginal swab, or any other biological material or biological fluid originally obtained from a human, animal, plant, or environmental sample.

In some embodiments, the amplifying of whole genome DNA in step (a) uses a DNA polymerase capable of producing high yields of purified DNA from the biological sample. In some embodiments, the polymerase is phi29 DNA polymerase or Bst polymerase.

In some embodiments, the amplifying of target DNA sequences in step (b) employs a polymerase chain reaction (PCR). In some embodiments, the unique DNA tag in step (b) comprises DNA sequences two or more bases long. In some embodiments, the target DNA sequence is selected from a human, microbial, animal, plant or viral gene sequence. In some embodiments, the target DNA sequence is selected from a 16S rRNA, 23S rRNA, eukaryotic 18S rRNA, human HLA, microbial toxin producing genes, microbial pathogenicity genes, microbial plasmid genes, human immune system genes, immune system components, ribosomal RNA genes, and other variable genetic regions of non-human organisms.

In some embodiments, the high throughput DNA sequencing in step (c) is a next-generation sequencing (NGS) method selected from: single-molecule real-time sequencing, ion semiconductor sequencing, pyrosequencing, sequencing by synthesis, sequencing by ligation and chain termination sequencing. In some embodiments, the separating in step (d) employs computer implemented methods.

Also disclosed herein are kits for identifying microbes, or other target DNA sequences, in a biological sample, comprising: (a) a DNA polymerase capable of producing high yields of purified DNA from a biological sample; (b) control samples and corresponding primers for amplifying target DNA sequences from the control samples; and (c) experimental primers for amplification of one or more microbe target DNA sequences from the biological sample. In some embodiments, the DNA polymerase is phi29. In some embodiments, the microbe target DNA sequence is a 16S rRNA gene sequence. In some embodiments, the experimental primers comprise unique DNA tags corresponding to the biological sample. In some embodiments, the kit further comprises one or more reagents to lyse cells so as to release DNA from cells in the sample. In some embodiments, such reagents are selected from the group consisting of: enzymes such as lysozyme or proteinase, a base such as KOH or NaOH, a detergent such as NP-40, CHAPS, Triton X-100, or sodium dodecyl sulfate.

### BRIEF DESCRIPTION OF THE DRAWINGS

FIG. 1 illustrates an embodiment of the present invention as described herein.

FIGS. 2A-B: FIG. 2A illustrates the analysis of 8 of the 96 PCR-amplified barcoded samples on a 0.8% agarose gel stained with ethidium bromide. A 2 kilobase (kb) DNA ladder is shown in the leftmost lane as a size reference. FIG. 2B is an image of 96 PCR-amplified barcoded samples from real-time PCR fluorescence monitoring of PCR product formation. Relative Fluorescence Units on the y-axis increases as PCR product is synthesized during PCR. The number of PCR cycles is shown on the x-axis. Each of the 96 samples has a different unique barcode, all of the amplifications have similar Cq between 20 and 23, as well as similar amplification profiles. The horizontal bar is represents the background threshold for Cq determination.

FIG. 3 illustrates a pooled sample gel where all PCR-amplified samples were pooled and purified using the SPRI protocol described in Example 3. Lane 1 contains a 2 kb DNA ladder, lane 2 shows the pooled PCR samples (1 microliter) and lane 3 shows the pooled PCR samples after SPRI purification (1 microliter).

FIG. 4 is a bar graph depicting the read lengths resulting from PacBio sequencing of the PCR sample pool (15 microliters; 2355 nanograms). The y-axis depicts the number of reads, the x-axis shows the read length. The most abundant read lengths were approximately 1500 bases, the size of the 16S rRNA amplicon.

FIG. 5 is a bar graph depicting an example PacBio RS II sequencing run after barcodes were demultiplexed and sorted by sample in silico.

FIG. 6 illustrates a dendrogram created from the sequencing data showing the microbes in the sample identified by genus on the right of the Figure. The lines represent the taxonomic evolutionary relationship between the microbes.

### DETAILED DESCRIPTION OF THE INVENTION

The subject matter that is regarded as the invention is particularly pointed out and distinctly claimed in the claims at the conclusion of the specification. The foregoing and other objects, features, and advantages of the invention will be apparent from the following detailed description taken in conjunction with the accompanying drawings and examples.

High throughput sequencing (HTS) platforms can sequence millions of DNA molecules in a single run, enabling mixing of many samples for simultaneous sequencing, taking advantage of the large numbers or reads produced by NGS systems to decrease the individual sample cost. Difficulties that must be overcome to realize full potential of HTS include highly parallelized sample purification, and sample identification for each read that enables samples to be combined for highly parallelized sequencing. The present invention utilizes multiple technologies to maximize the benefits of HTS for sequencing multiple DNA targets. First, crude DNA samples are prepared for downstream manipulation using a scalable method that works in the presence of contaminants (for example, phi29 whole genome amplification). DNA reads from each sample (for example, amplicons) are tracked by attaching unique DNA sequences (barcodes) to each sample during PCR. After barcoding, samples are mixed and sequenced together on a HTS platform, and the barcodes are used post-sequencing to sort reads by sample in silico. Together, these technologies are used in combination to streamline simultaneous handling of multiple samples in parallel across the sample preparation and sequencing workflow to decrease sample processing complexity and hands on time.

The combination of an amplification technique as a DNA preparation that works in the presence of contaminants that can be easily scaled to throughputs needed for subsequent sample barcoding/mixing followed by simultaneous NGS sequencing of 96 or more samples. The methods and kits disclosed herein combine genome amplification, DNA barcodes and NGS sequencing for targeted DNA sequencing and can be used to identify target DNA sequences, such as microbial gene sequences, in biological samples.

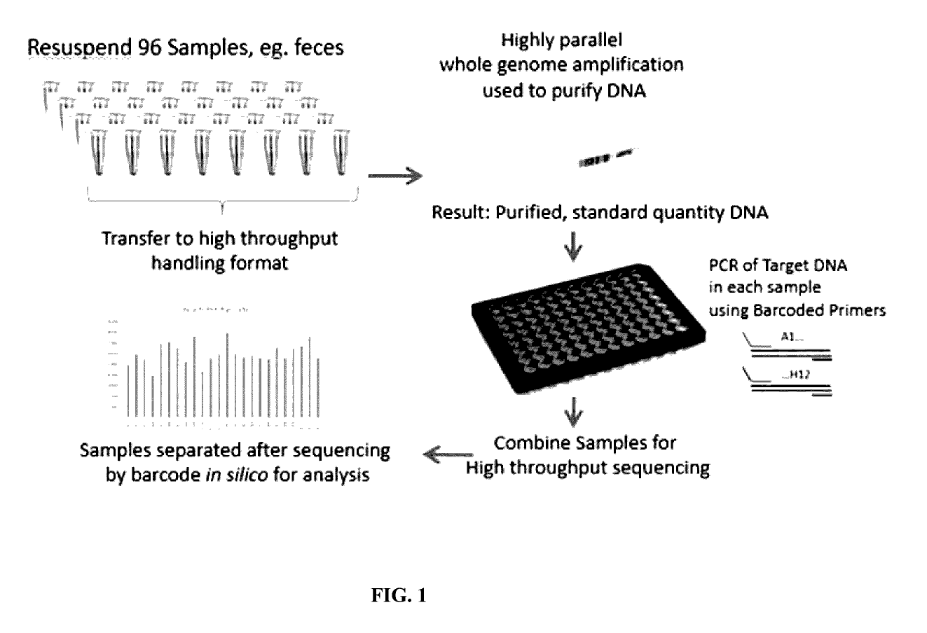

FIG. 1 illustrates one embodiment of the methods described herein where 96 crude samples (here, human feces) are collected in sample tubes then resuspended and transferred to a high throughput handling format (here, a 96-well multiwell plate). Each sample is then subject to whole genome amplification to produce standard yields of purified DNA, for example using alkaline lysis to open cells, followed by addition of phi29 or Bst DNA polymerase to amplify the DNA in the crude lysis. Next, barcoded target DNA sequences are added to the target sequence in each sample by PCR, utilizing primers specific for the target DNA and having a unique sequence tag specific for the corresponding sample. The barcoded PCR target DNA sequences are combined for high throughput sequencing. The sequencing results for each sample are separated by barcode and analyzed (here, using computer-implemented methods). Additional embodiments are further described herein.

Whole Genome Amplification

Samples: Biological samples may include, without limitation, feces, cell lysate, tissue, blood, tumor, tongue, tooth or, buccal swab, phlegm, mucous, wound swab, skin swab, vaginal swab, or any other biological material or biological fluid originally obtained from a human, animal, plant, or environmental sample. In some embodiments, the biological sample is crude or partially-purified. A “crude biological sample” as used herein, means a sample that has not been processed, altered or treated relative to its natural state. A “partially-purified biological sample” as used herein, means a sample that has been processed, altered or treated relative to its natural state but still contains contaminants or impurities.

To carry out the methods disclosed herein, biological samples may be transferred to multi-well plates, for example 8, 16, 24, 46, 96, 384 or 1536 well plates, or other vessels suitable for high-throughput analysis, or to microreactors contained in microfluidics devices that integrate one or more laboratory functions. In some embodiments, the high throughput format is selected from the group consisting of: at least eight samples, at least sixteen samples at least twenty-four samples, at least forty-eight samples, at least ninety-six samples, at least 384 samples or at least 1536 samples.

Cell lysis: The methods disclosed herein further comprise a step of cell lysis or cell membrane solubilization to open the cells to make the DNA accessible for amplification by a polymerase. Methods and reagents for cell lysis and cell membrane solubilization are known in the art, for example, alkaline lysis (Bimboim, H. C. and J. Doly, J., A rapid alkaline extraction procedure for screening recombinant plasmid DNA, Nucl. Acids Res. (1979) 7 (6): 1513-1523), detergent lysis, and enzymatic lysis (for example, lysozyme). For example, cells in a biological sample can be lysed in an alkaline solution consisting of about 0.2M potassium hydroxide (KOH). Either higher or lower concentrations of KOH may be used. Other bases, such as sodium hydroxide (NaOH), may also be used. In some embodiments, a detergent, such as sodium dodecyl sulfate, may be used to solubilize cell membranes and proteins. Thus, in some embodiments, the method comprises reagents to lyse cells or solubilize cell membranes so as to release DNA from cells in the sample, such as alkaline reagents or bases, for example potassium hydroxide or sodium hydroxide, or a detergent, such as sodium dodecyl sulfate (SDS), NP-40, CHAPS or Triton X-100. Cell lysis may also include mechanical methods for physical membrane disruption such as homogenization or grinding. The method(s) and reagent(s) used for cell lysis or cell membrane solubilization may be selected based on the cell type and the nature of the biological sample. Other considerations for optimal cell lysis include the buffer, pH, salt concentration and temperature, as well as the compatibility of the chosen detergent with downstream applications.

DNA polymerase: In some embodiments, amplifying whole genome DNA in step (a) employs a DNA polymerase capable of producing high yields of purified DNA from the crude biological sample. In some embodiments, the polymerase is a strand displacement DNA polymerase. In some embodiments, the polymerase is phi29 DNA polymerase (NCBI Accession No: ACE96023, U.S. Pat. Nos. 5,198,543 and 5,001,050). In some embodiments, the polymerase is selected from the group consisting of: phi29, Thermostable Bst DNA polymerase exonuclease (−) large fragment, Exonuclease (−) Bca DNA polymerase, Thermus aquaticus YT-1 polymerase, Phage M2 DNA polymerase, Phage PRD1 DNA polymerase, Exonuclease (−)VENT DNA polymerase, Klenow fragment of DNA polymerase I, T5 DNA polymerase, and PRD1 DNA polymerase.

The amino acid sequence of phi29 DNA polymerase is shown as SEQ ID NO:1:

(SEQ ID NO: 1)

MKHMPRKMYSCDFETTTKVEDCRVWAYGYMNIEDHSEYKIGNSLDEFMA
WVLKVQADLYFHNLKFDGAFIINWLERNGFKWSADGLPNTYNTIISRMG
QWYMIDICLGYKGKRKIHTVIYDSLKKLPFPVKKIAKDFKLTVLKGDID
YHKERPVGYKITPEEYAYIKNDIQIIAEALLIQFKQGLDRMTAGSDSLK
GFKDIITTKKFKKVFPTLSLGLDKEVRYAYRGGFTWLNDRFKEKEIGEG
MVFDVNSLYPAQMYSRLLPYGEPIVFEGKYVWDEDYPLHIQHIRCEFEL
KEGYIPTIQIKRSRFYKGNEYLKSSGGEIADLWLSNVDLELMKEHYDLY
NVEYISGLKFKATTGLFKDFIDKWTYIKTTSEGAIKQLAKLMLNSLYGK
FASNPDVTGKVPYLKENGALGFRLGEEETKDPVYTPMGVFITAWARYTT
ITAAQACYDRIIYCDTDSIHLTGTEIPDVIKDIVDPKKLGYWAHESTFK
RAKYLRQKTYIQDIYMKEVDGKLVEGSPDDYTDIKFSVKCAGMTDKIKK
EVTFENFKVGFSRKMKPKPVQVPGGVVLVDDTFTIK.

Phi29 polymerase is also useful because it can function in the presence of contaminants that strongly inhibit other polymerases. Crude or partially-purified biological samples with high levels of inhibitors can be processed using phi29 to produce target DNA that can be used directly in downstream reactions, which is very desirable in a high throughput environment. Phi29 DNA polymerase is commercially available from vendors such as Thermo Fisher Scientific (Waltham, Mass.) and New England BioLabs (Ipswich, Mass.).

For some research applications, the use of phi 29 amplification techniques may present difficulty because of bias for or against certain sequences across a genome. However, because the final 16S rRNA gene target in each microbe is very similar in sequence, the potential for bias is greatly reduced. In other words, since bias appears to be related to sequence context, in similar sequences such as 16S rRNA genes, bias is cancelled out, because the PCR target is similar in sequence composition and length for every source genome. This means that bias between genomic regions commonly seen for whole genome amplification techniques will be greatly reduced when phi 29 is used for assays comparing nearly identical targeted regions of DNA. As this disclosure outlines, specific amplicon targets such as the 16S rRNA gene are sequenced as a target, isothermal DNA amplification using phi29 DNA polymerase has attractive features as a rapid, low cost, automation friendly DNA purification method that can substitute for standard multi-step DNA preparation methods. It is not used as an amplification technique to acquire sufficient quantity of DNA for sequencing, because DNA is present in abundance in fecal samples, but as a purification technique. In particular, a single step phi29 isothermal incubation starting with whole cell DNA in the presence of high levels of contaminants produces DNA clean enough for PCR, sequencing and other analytical methods that would typically require multiple purification steps to remove contaminants and inhibitors. Because only one region of the genome is targeted in the subsequent targeted assay, context-specific amplification bias is not a problem the way it is for whole genome applications, where different regions of the genome can be amplified with 100×or more bias. Furthermore, the reaction can be designed to produce standard yields of output DNA, even with highly variable input. Benefits of standard yields from variable input are twofold; a simplified and more robust sample input protocol, and no need for DNA quantitation of output sample.

Rolling circle amplification protocols, materials, and methods using phi29 polymerase are described in U.S. Pat. No. 6,124,120, which is incorporated by reference herein in its entirety.

Advantages of using phi29 as a DNA purification technique are outlined in Deadman, R., and K. Jones. “DNA purification through amplification: Use of Phi29 DNA polymerase to prepare DNA for genomic analyses” Amersham Biosciences Life Science News 18 (2004): 14-15, which highlights the use of phi29 amplification for sequencing of single clones using Sanger sequencing, and mentions that “amplified DNA has been successfully used in many applications including PCR (simple, multiplex and real-time), SNP genotyping (Third Wave Invader™ assay (Third Wave Technologies, Inc., Madison, Wis.), MegaBACE™ SNuPe™ genotyping kit (GE Healthcare Life Sciences, Pittsburgh, Pa.), Affymetrix™ GeneChip™ HuSNP™ chip (Affymetrix, Santa Clara, Calif.), Pyrosequencing, STR and SSR genotyping, comparative genomic hybridization (CGH), cloning and library construction, heteroduplex analysis, slot and dot blots, yeast-2-hybrid systems, and microarray analysis.”

Other polymerases known in the art, may be used in the methods disclosed herein, including Thermostable Bst DNA polymerase exonuclease (−) large fragment (Aliotta, J. M., et al. Genet. Anal. 12:185-195, (1996)), Exonuclease (−) Bca DNA polymerase (Walker, G. T. and Linn, C. P. Clinical Chemistry 42:1604-1608 (1996)), thermus aquaticus YT-1 polymerase (Lawyer, F. C., et al. J. Biol. Chem., 264, 6427-6437 (1989)), Phage M2 DNA polymerase (Matsumoto, K., et al. Gene 84:247-255 (1989)), Phage PRD1 DNA polymerase (Jung, G., et al. Proc. Natl. Acad. Sci. U S. A. 84:8287-8291 (1987)), Exonuclease (−)VENT DNA polymerase (Kong, H., et al. J. Biol. Chem. 268:1965-1975 (1993)), Klenow fragment of DNA polymerase I (Jacobsen, H., et al. Eur J. Biochem, 45:623-627 (1974)), T5 DNA polymerase (Chatterjee D. K., et al. Gene 97:13-19 (1991); and U.S. Pat. No. 5,270,179), and PRD1 DNA polymerase (Zhu, W; Ito, J., Biochimica et Biophysica Acta 1219:267-276 (1994)).

A publication by Robert Pinard, et al. titled “Assessment of whole genome amplification-induced bias through high-throughput, massively parallel whole genome sequencing” (BMC Genomics 2006, 7:216) indicated that the bias across the genome for certain sequences was up to 100 fold, implying that there are severe limitations on the utility of the method for DNA purification. A publication demonstrating amplification of DNA from a single cell for 16S rRNA amplicon sequencing contains useful discussion of bias as it relates to number of cells in starting material. (Ragunathan, et al., Genomic DNA Amplification from a Single Bacterium, Appl. Environ. Microbiol. June 2005, vol. 71 no. 6 3342-3347)

Amplification: The methods disclosed herein include a step of amplifying whole genome DNA from biological samples in a high throughput format. Whole genome amplification steps may include thermocycling or isothermal protocols, or a combination thereof. Whole genome amplification primers may include random primers or target specific primers, or combinations thereof.

Whole genome DNA amplification techniques are typically employed when DNA sources are limited, with the goal of producing enough DNA for study. Non-limiting examples of amplification techniques known in the art include improved primer extension preamplification PCR (I-PEP-PCR), phi29 amplification (see U.S. Pat. No. 6,280,949, which is incorporated by reference herein in its entirety), and degenerate oligonucleotide primer (DOP) PCR.

DNA Barcodes and PCR

DNA barcodes allow objective identification of a sequence's sample of origin using a short section of artificially generated DNA sequence. Just as the unique pattern of bars in a universal product code (UPC) identifies each consumer product, a “DNA barcode” is a specially designed DNA sequence added during sample preparation that identifies the sample source for each read output by a DNA sequencer. DNA is extracted from each sample, and the unique identifying barcode for that sample is added during PCR of each sample. After each sample undergoes a separate PCR/barcoding process, multiple PCR amplicons can be mixed together (multiplexed) for sequencing, allowing many samples to be sequenced together, reducing hands on time and complexity. All DNA amplicons from all samples are sequenced simultaneously, and the sample of origin for each amplicon can be tracked using the barcodes, so all reads from a sample can be grouped together again post-sequencing.

DNA barcoding has been described for the very first of the NGS platforms, the GS 20, in 2007 (Parameswaran P, Jalili R, Tao L, et al. A pyrosequencing-tailored nucleotide barcode design unveils opportunities for large-scale sample multiplexing. Nucleic Acids Research. 2007; 35(19):e130). Since that time, barcoding strategies have been made commercially available by Illumina (San Diego, Calif.), Pacific Biosciences (Menlo Park, Calif.), and Thermo Fisher Scientific (Waltham, Mass.), such as the Ion Torrent™ (Thermo Fisher Scientific, Waltham, Mass.) sequencing platforms.

Barcoding individual samples with specific DNA tags allows many samples to be combined for sequencing, streamlining the workflow, reducing workflow complexity, decreasing time to result, and reducing costs. DNA barcodes can be selected to be of sufficient length to generate the desired number of barcodes with sufficient variability to account for common sequencing errors, generally ranging in size from about 2 to about 20 bases, but may be longer or shorter. Longer barcodes will permit higher sequence diversity and typically allow more samples to be combined. The target specific PCR sequences for the forward and reverse PCR primers can be specific for any DNA sequence, in coding or non-coding regions of a target genome, plasmid, or organelle.

The methods disclosed herein include a step of amplifying one or more target DNA sequences from the purified genomic DNA (as described above), wherein each targeted DNA sequence has a unique DNA tag corresponding to the sample. In some embodiments, amplification of target DNA sequences employs a polymerase chain reaction (PCR). Methods for conducting PCR are known in the art and also described in Example 2 herein and examples of PCR primers to amplify the bacterial 16S rRNA gene are included in Example 3, Table 3. In some embodiments, the target DNA sequence is an amplicon, or targeted gene sequence, such as bacterial 16S rRNA gene, 23S rRNA gene, eukaryotic 18S rRNA, human HLA, microbial toxin producing genes, microbial pathogenicity genes, microbial plasmid genes, human immune system genes, immune system components and other variable genetic regions of non-human organisms.

Sequencing

A high throughput sequencing method can be any sequencing method, with high throughput generally meaning greater than 1000 reads per run. Next-generation sequencing (NGS) refers to modem high throughput sequencing platforms that parallelize the sequencing process, producing thousands or millions of sequences concurrently, in contrast to less-efficient and more expensive standard dye-terminator methods. Non-limiting examples of NOS methods include single-molecule real-time sequencing (also referred to as Pacific Biosciences or PacBio), ion semiconductor (also referred to as Ion Torrent sequencing), pyrosequencing (also referred to as Roche 454), sequencing by synthesis (also referred to as Illumina sequencing), sequencing by ligation (also referred to as SOLiD sequencing) and chain termination sequencing (also referred to as Sanger sequencing).

High throughput DNA sequencing is carried out on the pooled, barcoded PCR amplicon DNA sequences as described above, producing a file with individual DNA sequences from all samples in random order. In some embodiments, the high throughput DNA sequencing is a next-generation sequencing (NGS) method, such as single-molecule real-time sequencing, ion semiconductor sequencing, pyrosequencing, sequencing by synthesis, sequencing by ligation and chain termination sequencing. The DNA sequences that originated with each sample are identified by barcode after sequencing and all reads are sorted into files by barcode. The DNA sequences from each sample are mapped to one of the many available databases containing microbial gene sequences to identify the microbes that are present in that sample. In some embodiments, separating the DNA sequences and identifying target DNA sequences employs computer implemented methods and database searches. In some embodiments, the databases may include online databases such as BLAST (Altschul, S. et al. (1990). “Basic local alignment search tool”. Journal of Molecular Biology. 215 (3): 403-410), GreenGenes (Appl. Environ. Microbiol. July 2006 vol. 72 no. 7 5069-5072), SILVA (Quast, et al., (2013) Nucl. Acids Res. 41 (D1): D590-D596.), and/or analysis pipelines such as QIIME (Caporaso I., Nature Methods, 2010; doi:10.1038/nmeth.f.303) or mothur (Schloss, et al., Appl. Environ. Microbiol. December 2009 vol. 75 no. 23 7537-7541).

Diagnostic Methods

The methods and kits described herein may be used or adapted for diagnostic purposes, such as the following:

16S rRNA high throughput sequencing diagnostics. Amplification used for DNA purification prior to parallel PCR of the 16S rRNA genes from all bacteria in fecal samples, followed by addition of barcodes and subsequent parallel shotgun sequencing to identify the microbes in the mixture. The methods described herein can be expanded to microbial DNA sources other than feces.

Other high-throughput targeted DNA diagnostics. Any genomic target that is amplified as part of complex source DNA can be expected to amplify with similar bias independent of the source genome. This makes whole genome amplification methods, which typically impart bias to the amplified sample, an ideal purification method for amplicon sequencing or similar diagnostics like sequence capture methods that depend on a specific target gene region from each independent genome. For example, if a given target region is amplified 100-fold above background during whole genome amplification, corresponding target regions in other genomes can be expected to undergo similar bias during amplification, facilitating direct comparison by quantitative PCR and sequencing based methods. For example, ratios of 16S gene sequences between organisms in a sample will be similar, even if the region tends to be over- or under-amplified compared to other parts of the genome. If the target was a cancer gene or viral polymerase gene, or other translated or untranslated DNA regions, the ratio of individual target sequences will be maintained between the amplified and unamplified samples, enabling downstream applications to take full advantage of the clonal nature of NGS methods.

### EXAMPLES

### Example 1

### DNA Preparation—96 Samples

Formulations and preparation of reagents (including Lysis Buffer 1, Lysis Buffer 2, and Lysis Buffer 3) and materials are further described in Example 3, including commercial sources of reagents and materials.

Lysis Protocol for 96 Samples

25 microliters of Lysis Buffer 1 was added to 96 tubes or 96 wells of a sample block for resuspending 96 samples at room temperature. Sample block filling was done using a repeater pipette or reagent trough with 20-200 microliters multichannel pipette.

Less than 4 milligrams of sample was removed by inserting inoculating loop into fecal material and withdrawing as cleanly as possible. For the purposes of this Example, more than 4 milligrams of sample may interfere with downstream steps; the assay may be used with as little at 0.1 milligram of material. The loop was transferred to the appropriate tube or position in the 96 well block. The loop was twisted to transfer the sample into the 25 microliters of Lysis Buffer 1 in the sample block.

Loops were left in the block until each row was complete, then the row was marked and the loops in that row were removed, and the same marking procedure was used for the remaining rows to keep track of which samples were added and which one was next. 225 microliters of water was added to each well (e.g., sterile, purified, molecular biology grade water) using repeater pipette. The sample block was covered with an adhesive lid and shaken for a few seconds on vortexer at very low speed to avoid splashing. The block was spun briefly at 500 rpm as needed to remove drops from lid, to avoid contamination of adjacent wells, then the lid was removed.

The samples were allowed to settle for at least 1 minute at room temperature while the 96 well lysis plate was prepared for the next step. The sample is stable for at least 2 hours at room temperature.

While samples settled in Lysis Buffer 1, a second 96 well lysis plate was prepared by adding 6 microliters Lysis Buffer 2 to each well of an empty 96 well plate using a repeater pipette or reagent trough with 0.5-10 microliter multichannel pipette.

2 microliters resuspended fecal sample was transferred from the first plate into the second plate containing 6 microliters Lysis Buffer 2 for all 96 samples using a multichannel pipette, avoiding settled debris. The samples were mixed by pipetting up and down.

Samples were incubated for 5 minutes at room temperature after sample transfer to the second plate was complete. Incubation can be extended to 10 minutes as needed.

10 microliters of Lysis Buffer 3 was added to all 96 samples using a repeater pipette, or reagent trough with 0.5-10 microliter multichannel pipette.

The completed Lysis Mix was incubated on ice and used in the Purification Protocol within 24 hours. The plate was covered if stored for an extended period of time.

If bubbles were noticed at or near the bottom of the wells in the plate, the plate was spun at 2000×g in a plate centrifuge for 30 seconds to remove bubbles, prior to commencing the Purification Protocol described below.

Purification Protocol for 96 Samples

The Purification Buffer Mix was thawed and placed on ice with the Purification Enzyme Mix. The Purification Mix was prepared by adding the entire contents of the Purification Buffer Mix to the Purification Enzyme Mix. The Purification Buffer Mix contained 860 microliters volume and the Purification Enzyme Mix contained 40 microliters volume; they were mixed by pipetting up and down and stored on ice. 9 microliters of Purification Mix was dispensed into each of the 96 wells of a new 96 well plate at 4° C.

Using a 0.5-10 microliter multichannel pipette, 1 microliter of each of the 96 Lysis Mix samples (as described above) was transferred to the corresponding wells containing Purification Mix. Samples were mixed by pipetting up and down.

The plate was covered with PCR film and incubated at 30° C. for 90 minutes on hotplate or thermocycler. Incubation can run up to 22 hours at 30° C. without affecting results. Longer incubations (up to 18 hours) can result in higher DNA yields, which can improve PCR. Completed reaction can be stored at 4° C. overnight, if needed. Long term storage of DNA beyond 2 days requires treatment with Proteinase K to prevent degradation of the DNA (see Proteinase K protocol in Example 3). For fastest workflow, the next step (96 well plate containing PCR Premix) was prepared about 30 minutes prior to end of incubation. If bubbles were noticed at or near the bottom of the wells in the plate, the plate was spun at 2000×g in a plate centrifuge for 30 seconds to remove bubbles. Purified samples were then ready for PCR as described in Example 2, below.

### Example 2

### 16S rRNA Amplicon

PCR Protocol—Adding Unique Barcodes to 96 Samples

Approximately 30 minutes before needed, the PCR mix was prepared on ice. 200 microliters of water was added to the PCR Premix tube containing 319 microliters of PCR Premix. The solution was pipetted up and down to mix. 5 microliters of PCR mix was dispensed onto the side of each of the 96 wells in the 96 Well Plate with Barcoded Primers (primer sequences are listed in Table 3 of Example 3, below) on ice (using a repeater pipette). In a prepared plate, the primers are in the blue dot at the bottom of the well, so the PCR premix was not dispensed directly at the bottom of the well to avoid barcode cross-contamination. The plate was tapped so the PCR Premix fell to the bottom of each well. The plate was incubated on ice for about 15 minutes to ensure primers with blue dye dissolve. Samples were checked after 5 minutes to ensure that all wells had some blue color. As needed, samples were pipetted up and down to dissolve the primers. 1.0 microliter of purified sample ready for PCR was added on ice (using a multichannel pipette). Samples were pipetted up and down when added to mix.

Samples were checked to ensure that samples were blue and had uniform color from well to well. If blue color was not uniform, samples were tapped gently while samples remained chilled. Taq polymerase that was used in this PCR reaction was not hot start, and warming the reaction to room temperature can reduce PCR efficiency. In some embodiments, hot start PCR polymerases can be used.

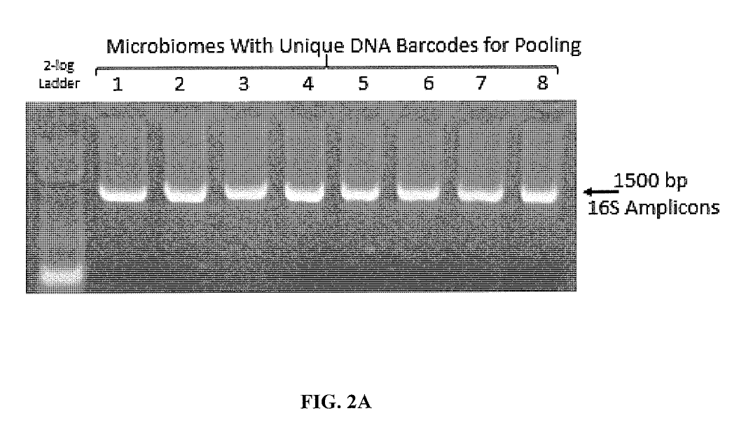

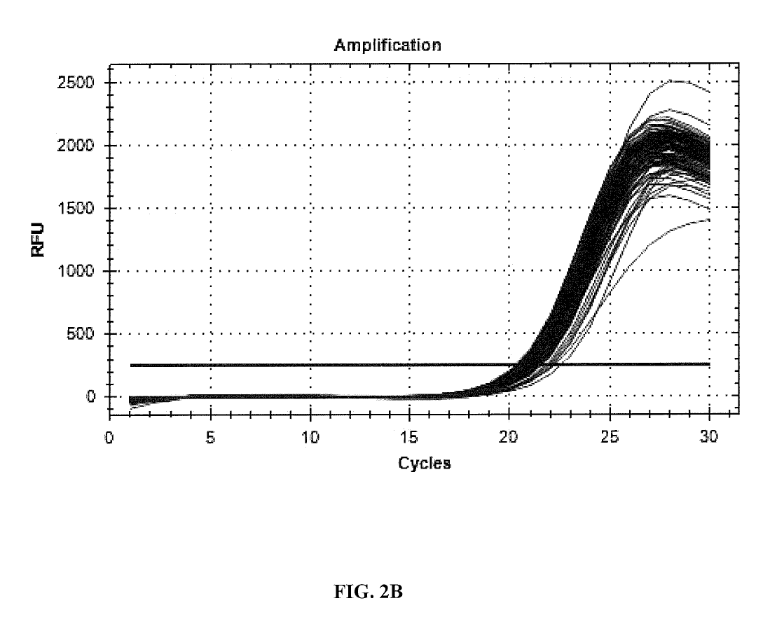

With the reaction kept on ice, 10 microliters of room temperature Mineral Oil was added using a repeater pipette. Samples were not spun down because spinning can warm the plate and negatively impact PCR yield. The plate was sealed by adding PCR film, on ice. The PCR program was started, and a heated lid was used as available. When the block temperature reached 60° C., the PCR plate was removed from ice and places directly on the PCR machine, and the lid was closed. The following PCR program parameters were used: (1) 95° C. for 60 seconds; (2) 30 cycles of: 95° C. for 20 seconds, 51° C. for 45 seconds, 72° C. for 1 minute 30 seconds; (3) final 72° C. extension for 3 minutes; (4) hold at 4° C. The following optional step was used for quality control: when the PCR reaction was complete, 1.5 microliters of each reaction was removed for visualization on 0.8% agarose gel. As shown in FIG. 2, a single band at 1500 base pairs (bp) was visible for all 96 samples, indicating the presence of 16S rRNA target DNA.

All 96 barcoded PCR samples were combined into single tube to create one 96-plex sample. Using a multichannel pipette, the blue aqueous sample was removed from the bottom of the tube into an 8-strip PCR microfuge tube, leaving most of the clear mineral oil behind. Some oil carry over into the pool is acceptable in order to get all the PCR reaction from the well. When the PCR product was removed from the 96 well plate, the samples in the 8-strip tubes were combined into a 1.5 milliliter microfuge tube. The final volume of pooled PCR product was around 500 microliters and was stored at 4° C.

SPRI purification was performed on 100 microliter blue PCR product at 1:1 according to the protocol described in Example 3, below, to remove residual primers and small products from the sample. The sample was stored at 4° C. The final concentration of DNA was measured by OD 260 or PicoGreen assay.

The sample was ready for sequencing library preparation. The sample was stored at 4° C. Freezing the sample can damage the DNA and should be avoided.

Results

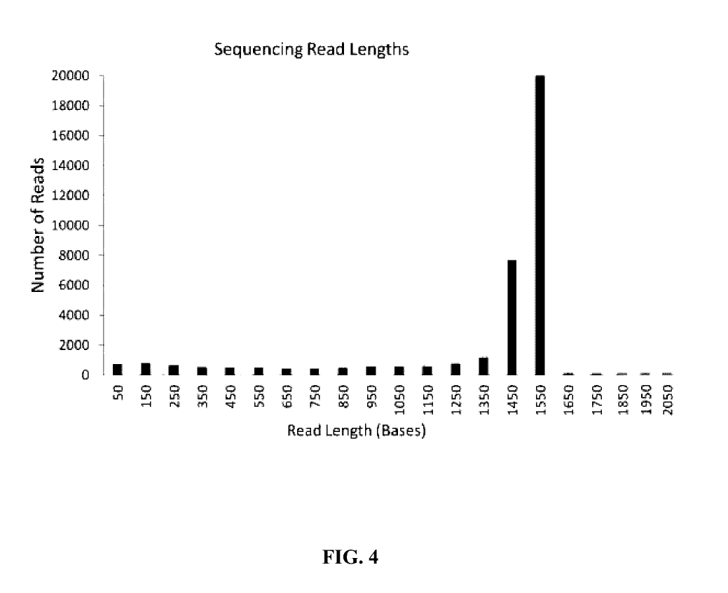

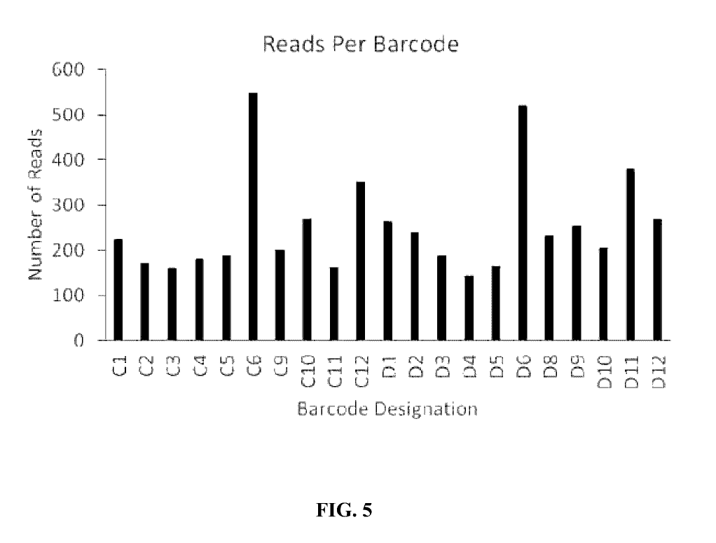

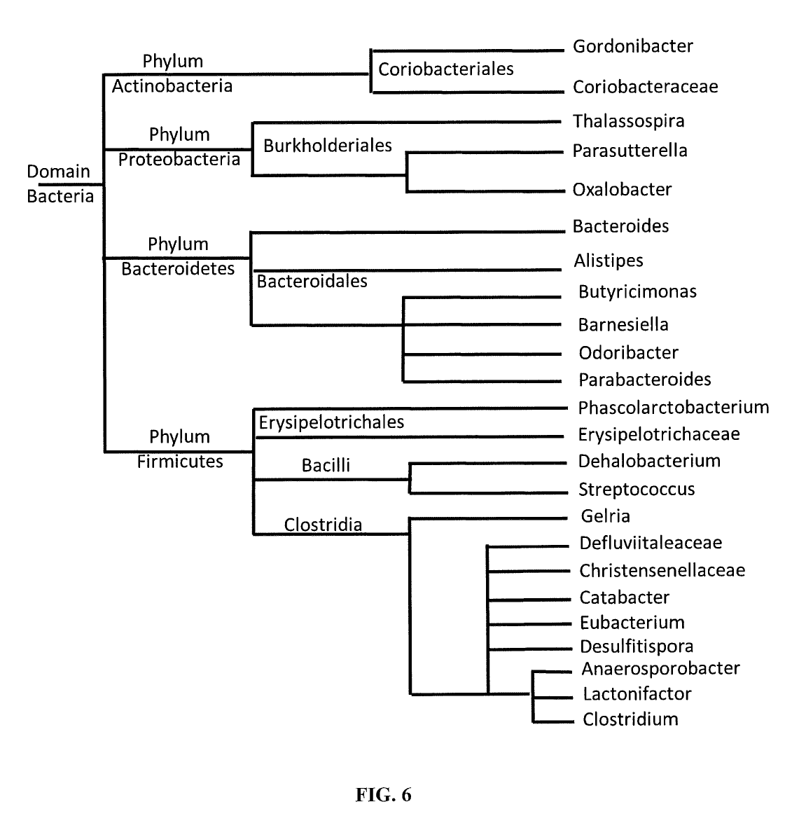

The methods and reagents described in the Examples were tested in a functional assay using ninety-six repeats from a crude sample of frozen human feces; each sample was independently pulled from the same sample tube. Two microliters of each PCR product was analyzed on 0.8% agarose gel stained with ethidium bromide and a 1500 base pair 16S rRNA band was visible in each lane on 0.8% agarose gel as shown in FIG. 2. Therefore, 95/96 repeats yielded PCR product. One repeat yielded no product, two bands were light with lower product yield. PacBio sequencing was carried out on the samples as per manufacturer's instructions and most reads were at or near 1509 base pairs in length (FIG. 4), as expected for the 16S rRNA sequence. An example sequencing run shown in FIG. 5 averaged approximately 100 reads per barcode. Higher throughput runs could be expected to yield far more reads per barcode. The DNA barcodes were identified and sorted by barcode in silico using proprietary algorithms that search for each 16 base barcode at the beginning and end of each read. The sequenced reads were mapped to the GreenGenes database, where sequence matches identified multiple microbes per sample. The microbial identities output by GreenGenes were used as input to publically available FigTree software to create the dendrogram in FIG. 6 to visualize the relationships between the microbes that were present in the biological sample. The dendrogram shows the identity of the microbes in the test microbiome, with evolutionary relationships to other organisms in the population. Similar dendrograms can be generated for each of the 96 samples in the run.

### Example 3

### Materials, Reagents and Additional Methods

The materials, reagents and methods used in Examples 1 and 2 are described in further detail in this Example 3.

Reagents for DNA Preparation—96 Samples:

Lysis Buffer 1 (0.5% SDS): 25 microliters were used per sample. To prepare enough for 104 samples, 1.25 grams of SDS was dissolved in 250 milliliters of water.

Lysis Buffer 2 (0.2M KOH): 6 microliters was needed per reaction (576 microliters total). To prepare 0.2M KOH (0.01122 g/ml, FW 56.11), 0.337 grams KOH (Fisher) was dissolved in 30 milliliters of water.

Lysis Buffer 3 (Neutralization Solution): 10 microliters were needed per reaction (960 microliters total). New 500mM Tris pH 7.5 was made using Tris acid and Tris base. For 100 milliliters of 0.5M, 6.35 grams TrisHCl and 1.18 grams TrisBase were mixed and the pH was measured using pH paper (pH˜7.5). Table 1 below lists phi 29 reagents for 100×10 microliter reactions:

| 2X phi29 | 100 reactions |
|---|---|
| H2O | 610 |
| 10x phi29 DNA Pol Reaction Buffer | 100 |
| 10 mg/ml BSA | 20 |
| dNTP mix, 10 mM each dNTP | 100 |
| Random Hexamers | 50 |
| Lysis Mix | 100 |
| Yeast Pyrophosphatase | 10 |
| phi29 DNA polymerase | 10 |
| Total volume for 10 ul reactions | 1000 |

Purification Enzyme Mix—10 microliters of Yeast Inorganic pyrophosphatase (100 units/ml), 10 microliters phi29 DNA polymerase (10,000 units/ml), 20 microliters of 10 mg/ml bovine serum albumin, and 20 microliters Diluent F were combined into one tube.

Purification Buffer Mix (860 microliters total)—Diluent F (60 microliters total)—1×Buffer Components: 100mM KCl, 10 mM Tris-HCl, 1 mM DTT, 0.1 mM EDTA, 0.5% Tween® 20 (Sigma-Aldrich Co. LLC, St. Louis, Mo.), 0.5% IGEPAL® CA-630, 50% Glycerol, pH 7.4@25° C.

610 microliters water, 10×reaction buffer, dNTPs (8 micromoles each dNTP), 100 uM random hexamers were combined in one tube.

phi29 DNA polymerase Reaction Buffer—1×Buffer Components: 50mM Tris-HCl, 10 mM MgCl2, 10 mM (NH4)2SO4, 4mM DTT, pH 7.5@25° C.

Random Hexamers: DNA Sequence 5′-NNNN*N*N-3′, where ‘*’ denotes a phosphorothiolate bond.

Lysis mix was added, 1 microliter per reaction, from lysis mix and stored at −20C.

Reagents for 16S rRNA Amplicon—96 Samples:

PCR Oil—mineral oil, light, available from commercial sources (for example, Fisher O121-1, Saybolt viscosity 158 max).

PCR Premix—104 reactions−3 microliters per 6 microliter reaction−312 microliters+6.864 microliters extra Taq=319 microliters

TABLE 2
PCR Recipe for Dried Primer at 6 microliters Per Reaction & 1 microliter
Template
Use New 1 microliter template for 6 microliter PCR Mix Recipe.

| | | | Volume μLs |
|---|---|---|---|
| | Volume μLs for | Number of | Needed for |
| | One Reaction | Reactions | Desired Reactions |
| NEB 2X Master Mix | 3.000 | 104 | 312 |
| Molecular Grade | 1.934 | 104 | 201.136 |
| Water | | | |
| Template | 1.000 | 104 | 104 |
| Taq Polymerase | 0.066 | 104 | 6.864 |
| (5000 U/mL) | | | |
| Total Volume | 6.00 | | 624 |

Water—201 microliters needed for 104 reactions.

PCR plate contained 2 microliters of 0.625 uM PCR forward and reverse primers (see Table 3, below), dried down prior to use.

PCR 2×Master Mix: 1×Master Mix Composition contained 10 mM Tris-HCl, 50 mM KCl, 1.5 mM MgCl2, 0.2 mM dNTPs, 5% Glycerol, 0.08% IGEPAL® CA-630, 0.05% Tween® 20, 25 units/ml Taq DNA Polymerase, pH 8.6@25° C.

Table 3 lists the PCR oligomer sequences of the forward and reverse Barcoded Primers described in Example 2:

PCR Oligomer Sequences

| Well | | |
|---|---|---|
| Position | Forward | Reverse |
| A1 | TCAGACGATGCGTCATAGAGTTTGATCMTGGCTCAG | CGATCAGCTGAGCGCGTACGGYTACCTTGTTACGACTT |
| A2 | GCGCGATACGATGACTAGAGTTTGATCMTGGCTCAG | ATCTAGCGTAGTGATGTACGGYTACCTTGTTACGACTT |
| A3 | TCAGACGATGCGTCATAGAGTTTGATCMTGGCTCAG | AGTGTGTCATGCGTGTTACGGYTACCTTGTTACGACTT |
| A4 | CTATACATGACTCTGCAGAGTTTGATCMTGGCTCAG | CGATCAGCTGAGCGCGTACGGYTACCTTGTTACGACTT |
| A5 | TGTGTATCAGTACATGAGAGTTTGATCMTGGCTCAG | TGCATGCACAGATGCGTACGGYTACCTTGTTACGACTT |
| A6 | GATCTCTACTATATGCAGAGTTTGATCMTGGCTCAG | GAGAGACGATCACATATACGGYTACCTTGTTACGACTT |
| A7 | GATCTCTACTATATGCAGAGTTTGATCMTGGCTCAG | AGATATGCGCGACACGTACGGYTACCTTGTTACGACTT |
| A8 | TACTAGAGTAGCACTCAGAGTTTGATCMTGGCTCAG | AGTGTGTCATGCGTGTTACGGYTACCTTGTTACGACTT |
| A9 | TGTGTATCAGTACATGAGAGTTTGATCMTGGCTCAG | TATGCATGACTGATATTACGGYTACCTTGTTACGACTT |
| A10 | TGTGTATCAGTACATGAGAGTTTGATCMTGGCTCAG | AGTGTGTCATGCGTGTTACGGYTACCTTGTTACGACTT |
| A11 | ACACGCATGACACACTAGAGTTTGATCMTGGCTCAG | TCTGTAGTGCGTGCGCTACGGYTACCTTGTTACGACTT |
| A12 | ACACGCATGACACACTAGAGTTTGATCMTGGCTCAG | AGTGTGTCATGCGTGTTACGGYTACCTTGTTACGACTT |
| B1 | TGTGTATCAGTACATGAGAGTTTGATCMTGGCTCAG | GTCGCGACGTCAGTGTTACGGYTACCTTGTTACGACTT |
| B2 | GATCTCTACTATATGCAGAGTTTGATCMTGGCTCAG | AGTGTGTCATGCGTGTTACGGYTACCTTGTTACGACTT |
| B3 | ACAGTCTATACTGCTGAGAGTTTGATCMTGGCTCAG | CGATCAGCTGAGCGCGTACGGYTACCTTGTTACGACTT |
| B4 | ACAGTCTATACTGCTGAGAGTTTGATCMTGGCTCAG | TCTGTAGTGCGTGCGCTACGGYTACCTTGTTACGACTT |
| B5 | ACAGTCTATACTGCTGAGAGTTTGATCMTGGCTCAG | AGTGTGTCATGCGTGTTACGGYTACCTTGTTACGACTT |
| B6 | ATGATGTGCTACATCTAGAGTTTGATCMTGGCTCAG | CGATCAGCTGAGCGCGTACGGYTACCTTGTTACGACTT |
| B7 | ATGATGTGCTACATCTAGAGTTTGATCMTGGCTCAG | TCTGTAGTGCGTGCGCTACGGYTACCTTGTTACGACTT |
| B8 | ATGATGTGCTACATCTAGAGTTTGATCMTGGCTCAG | AGTGTGTCATGCGTGTTACGGYTACCTTGTTACGACTT |
| B9 | CTGCGTGCTCTACGACAGAGTTTGATCMTGGCTCAG | TCTGTAGTGCGTGCGCTACGGYTACCTTGTTACGACTT |
| B10 | CTGCGTGCTCTACGACAGAGTTTGATCMTGGCTCAG | AGTGTGTCATGCGTGTTACGGYTACCTTGTTACGACTT |
| B11 | GCGCGATACGATGACTAGAGTTTGATCMTGGCTCAG | TCTGTAGTGCGTGCGCTACGGYTACCTTGTTACGACTT |
| B12 | GCGCGATACGATGACTAGAGTTTGATCMTGGCTCAG | AGTGTGTCATGCGTGTTACGGYTACCTTGTTACGACTT |
| C1 | CGCGCTCAGCTGATCGAGAGTTTGATCMTGGCTCAG | CGATCAGCTGAGCGCGTACGGYTACCTTGTTACGACTT |
| C2 | CGCGCTCAGCTGATCGAGAGTTTGATCMTGGCTCAG | TCTGTAGTGCGTGCGCTACGGYTACCTTGTTACGACTT |
| C3 | GCGCACGCACTACAGAAGAGTTTGATCMTGGCTCAG | TCTGTAGTGCGTGCGCTACGGYTACCTTGTTACGACTT |
| C4 | GCGCACGCACTACAGAAGAGTTTGATCMTGGCTCAG | AGTGTGTCATGCGTGTTACGGYTACCTTGTTACGACTT |
| C5 | ACACTGACGTCGCGACAGAGTTTGATCMTGGCTCAG | TCTGTAGTGCGTGCGCTACGGYTACCTTGTTACGACTT |
| C6 | ACACTGACGTCGCGACAGAGTTTGATCMTGGCTCAG | AGTGTGTCATGCGTGTTACGGYTACCTTGTTACGACTT |
| C7 | ACAGTCTATACTGCTGAGAGTTTGATCMTGGCTCAG | AGATATGCGCGACACGTACGGYTACCTTGTTACGACTT |
| C8 | ATAGAGACTCAGAGCTAGAGTTTGATCMTGGCTCAG | AGATATGCGCGACACGTACGGYTACCTTGTTACGACTT |
| C9 | ATAGAGACTCAGAGCTAGAGTTTGATCMTGGCTCAG | TCTGTAGTGCGTGCGCTACGGYTACCTTGTTACGACTT |
| C10 | ATAGAGACTCAGAGCTAGAGTTTGATCMTGGCTCAG | AGTGTGTCATGCGTGTTACGGYTACCTTGTTACGACTT |
| C11 | TAGATGCGAGAGTAGAAGAGTTTGATCMTGGCTCAG | TCTGTAGTGCGTGCGCTACGGYTACCTTGTTACGACTT |
| C12 | TAGATGCGAGAGTAGAAGAGTTTGATCMTGGCTCAG | AGTGTGTCATGCGTGTTACGGYTACCTTGTTACGACTT |
| D1 | TGTGTATCAGTACATGAGAGTTTGATCMTGGCTCAG | ATCTAGCGTAGTGATGTACGGYTACCTTGTTACGACTT |
| D2 | CACGCACACACGCGCGAGAGTTTGATCMTGGCTCAG | AGTGTGTCATGCGTGTTACGGYTACCTTGTTACGACTT |
| D3 | CGAGCACGCGCGTGTGAGAGTTTGATCMTGGCTCAG | CGATCAGCTGAGCGCGTACGGYTACCTTGTTACGACTT |
| D4 | CGAGCACGCGCGTGTGAGAGTTTGATCMTGGCTCAG | TCTGTAGTGCGTGCGCTACGGYTACCTTGTTACGACTT |
| D5 | ACACGCATGACACACTAGAGTTTGATCMTGGCTCAG | TGCATGCACAGATGCGTACGGYTACCTTGTTACGACTT |
| D6 | ACAGTCTATACTGCTGAGAGTTTGATCMTGGCTCAG | GAGAGACGATCACATATACGGYTACCTTGTTACGACTT |
| D7 | GAGACTCTGTGCGCGTAGAGTTTGATCMTGGCTCAG | AGTGTGTCATGCGTGTTACGGYTACCTTGTTACGACTT |
| D8 | GCTCGACTGTGAGAGAAGAGTTTGATCMTGGCTCAG | AGTGTGTCATGCGTGTTACGGYTACCTTGTTACGACTT |
| D9 | GATCTCTACTATATGCAGAGTTTGATCMTGGCTCAG | TATGCATGACTGATATTACGGYTACCTTGTTACGACTT |
| D10 | TACGACTACATATCAGAGAGTTTGATCMTGGCTCAG | AGTGTGTCATGCGTGTTACGGYTACCTTGTTACGACTT |
| D11 | GTCAGCTAGTGTCAGCAGAGTTTGATCMTGGCTCAG | AGTGTGTCATGCGTGTTACGGYTACCTTGTTACGACTT |
| D12 | GTGCAGTGATCGATGAAGAGTTTGATCMTGGCTCAG | TCTGTAGTGCGTGCGCTACGGYTACCTTGTTACGACTT |
| E1 | GTGCAGTGATCGATGAAGAGTTTGATCMTGGCTCAG | AGTGTGTCATGCGTGTTACGGYTACCTTGTTACGACTT |
| E2 | TGACTCGCTCATAGTCAGAGTTTGATCMTGGCTCAG | TCTGTAGTGCGTGCGCTACGGYTACCTTGTTACGACTT |
| E3 | TGACTCGCTCATAGTCAGAGTTTGATCMTGGCTCAG | AGTGTGTCATGCGTGTTACGGYTACCTTGTTACGACTT |
| E4 | CGCGCTCAGCTGATCGAGAGTTTGATCMTGGCTCAG | ATCTAGCGTAGTGATGTACGGYTACCTTGTTACGACTT |
| E5 | ATGCTGATGACGCGCTAGAGTTTGATCMTGGCTCAG | AGTGTGTCATGCGTGTTACGGYTACCTTGTTACGACTT |
| E6 | GACAGCATCTGCGCTCAGAGTTTGATCMTGGCTCAG | CGATCAGCTGAGCGCGTACGGYTACCTTGTTACGACTT |
| E7 | GACAGCATCTGCGCTCAGAGTTTGATCMTGGCTCAG | TCTGTAGTGCGTGCGCTACGGYTACCTTGTTACGACTT |
| E8 | TAGATGCGAGAGTAGAAGAGTTTGATCMTGGCTCAG | AGATATGCGCGACACGTACGGYTACCTTGTTACGACTT |
| E9 | GATCTCTACTATATGCAGAGTTTGATCMTGGCTCAG | CGAGACTGTCGATCTCTACGGYTACCTTGTTACGACTT |
| E10 | CGCGCTCAGCTGATCGAGAGTTTGATCMTGGCTCAG | CGAGACTGTCGATCTCTACGGYTACCTTGTTACGACTT |
| E11 | TCGATATACGACGTGCAGAGTTTGATCMTGGCTCAG | AGTGTGTCATGCGTGTTACGGYTACCTTGTTACGACTT |
| E12 | GATCGACTCGAGCATCAGAGTTTGATCMTGGCTCAG | AGTGTGTCATGCGTGTTACGGYTACCTTGTTACGACTT |
| F1 | CTGCGTGCTCTACGACAGAGTTTGATCMTGGCTCAG | ATCTAGCGTAGTGATGTACGGYTACCTTGTTACGACTT |
| F2 | CGTGCACATCTATAGCAGAGTTTGATCMTGGCTCAG | AGTGTGTCATGCGTGTTACGGYTACCTTGTTACGACTT |
| F3 | GACTGCACATGCACGAAGAGTTTGATCMTGGCTCAG | TCTGTAGTGCGTGCGCTACGGYTACCTTGTTACGACTT |
| F4 | GACTGCACATGCACGAAGAGTTTGATCMTGGCTCAG | AGTGTGTCATGCGTGTTACGGYTACCTTGTTACGACTT |
| F5 | ACAGTCTATACTGCTGAGAGTTTGATCMTGGCTCAG | TGCATGCACAGATGCGTACGGYTACCTTGTTACGACTT |
| F6 | TCGTCATACGCTCTAGAGAGTTTGATCMTGGCTCAG | AGTGTGTCATGCGTGTTACGGYTACCTTGTTACGACTT |
| F7 | CGACTACGTACAGTAGAGAGTTTGATCMTGGCTCAG | AGTGTGTCATGCGTGTTACGGYTACCTTGTTACGACTT |
| F8 | GCGTAGACAGACTACAAGAGTTTGATCMTGGCTCAG | AGTGTGTCATGCGTGTTACGGYTACCTTGTTACGACTT |
| F9 | CTGCGCAGTACGTGCAAGAGTTTGATCMTGGCTCAG | AGTGTGTCATGCGTGTTACGGYTACCTTGTTACGACTT |
| F10 | ATAGAGACTCAGAGCTAGAGTTTGATCMTGGCTCAG | CGAGACTGTCGATCTCTACGGYTACCTTGTTACGACTT |
| F11 | CTGATGCGCGCTGTACAGAGTTTGATCMTGGCTCAG | AGTGTGTCATGCGTGTTACGGYTACCTTGTTACGACTT |
| F12 | GCACATACACGCTCACAGAGTTTGATCMTGGCTCAG | AGTGTGTCATGCGTGTTACGGYTACCTTGTTACGACTT |
| G1 | AGAGAGAGACATGCGCAGAGTTTGATCMTGGCTCAG | AGTGTGTCATGCGTGTTACGGYTACCTTGTTACGACTT |
| G2 | ACTCTCGCTCTGTAGAAGAGTTTGATCMTGGCTCAG | AGTGTGTCATGCGTGTTACGGYTACCTTGTTACGACTT |
| G3 | GTACATATGCGTCTGTAGAGTTTGATCMTGGCTCAG | AGTGTGTCATGCGTGTTACGGYTACCTTGTTACGACTT |
| G4 | TGCTCGCAGTATCACAAGAGTTTGATCMTGGCTCAG | AGTGTGTCATGCGTGTTACGGYTACCTTGTTACGACTT |
| G5 | CTGTGTGTGATAGAGTAGAGTTTGATCMTGGCTCAG | AGTGTGTCATGCGTGTTACGGYTACCTTGTTACGACTT |
| G6 | CAGTGAGAGCGCGATAAGAGTTTGATCMTGGCTCAG | TCTGTAGTGCGTGCGCTACGGYTACCTTGTTACGACTT |
| G7 | CAGTGAGAGCGCGATAAGAGTTTGATCMTGGCTCAG | AGTGTGTCATGCGTGTTACGGYTACCTTGTTACGACTT |
| G8 | CATAGCGACTATCGTGAGAGTTTGATCMTGGCTCAG | AGTGTGTCATGCGTGTTACGGYTACCTTGTTACGACTT |
| G9 | CATCACTACGCTAGATAGAGTTTGATCMTGGCTCAG | AGTGTGTCATGCGTGTTACGGYTACCTTGTTACGACTT |
| G10 | CGCATCTGTGCATGCAAGAGTTTGATCMTGGCTCAG | AGTGTGTCATGCGTGTTACGGYTACCTTGTTACGACTT |
| G11 | TATGTGATCGTCTCTCAGAGTTTGATCMTGGCTCAG | TCTGTAGTGCGTGCGCTACGGYTACCTTGTTACGACTT |
| G12 | GTACACGCTGTGACTAAGAGTTTGATCMTGGCTCAG | AGTGTGTCATGCGTGTTACGGYTACCTTGTTACGACTT |
| H1 | CGTGTCGCGCATATCTAGAGTTTGATCMTGGCTCAG | TCTGTAGTGCGTGCGCTACGGYTACCTTGTTACGACTT |
| H2 | CGTGTCGCGCATATCTAGAGTTTGATCMTGGCTCAG | AGTGTGTCATGCGTGTTACGGYTACCTTGTTACGACTT |
| H3 | ATATCAGTCATGCATAAGAGTTTGATCMTGGCTCAG | TCTGTAGTGCGTGCGCTACGGYTACCTTGTTACGACTT |
| H4 | ATAGAGACTCAGAGCTAGAGTTTGATCMTGGCTCAG | ATCTAGCGTAGTGATGTACGGYTACCTTGTTACGACTT |
| H5 | CGCTGCGAGAGACAGTAGAGTTTGATCMTGGCTCAG | TCTGTAGTGCGTGCGCTACGGYTACCTTGTTACGACTT |
| H6 | TGCTCTCGTGTACTGTAGAGTTTGATCMTGGCTCAG | TCTGTAGTGCGTGCGCTACGGYTACCTTGTTACGACTT |
| H7 | CACTCGTGCACGATGCAGAGTTTGATCMTGGCTCAG | AGTGTGTCATGCGTGTTACGGYTACCTTGTTACGACTT |
| H8 | GAGATACGCTGCAGTCAGAGTTTGATCMTGGCTCAG | AGTGTGTCATGCGTGTTACGGYTACCTTGTTACGACTT |
| H9 | ACAGTCTATACTGCTGAGAGTTTGATCMTGGCTCAG | CGAGACTGTCGATCTCTACGGYTACCTTGTTACGACTT |
| H10 | ATCTCGAGATGTAGCGAGAGTTTGATCMTGGCTCAG | TCTGTAGTGCGTGCGCTACGGYTACCTTGTTACGACTT |
| H11 | ATCTCGAGATGTAGCGAGAGTTTGATCMTGGCTCAG | AGTGTGTCATGCGTGTTACGGYTACCTTGTTACGACTT |
| H12 | ACGATCACTCGTGTCAAGAGTTTGATCMTGGCTCAG | AGTGTGTCATGCGTGTTACGGYTACCTTGTTACGACTT |

Protocol for SPRI Purification of PCR Pool:

1. Agencourt Ampure XP (#A63880, Beckman Coulter) beads were used to purify the 16S amplicon pool as per manufacturer's instructions.

2. OD of 2 microliters was measured in 98 microliters water or fluorescence-based quantitation was carried out. 100 microliters SPRI purification yielded 50 microliters of PCR product at ˜120 ng/ul, for a total of ˜6 micrograms PCR product. SPRI can be repeated on additional aliquots, or scaled up, if more product is needed. 96-well process yielded around 500 microliters of pool total, so up to 5×6 micrograms or 30 micrograms PCR product can be obtained.

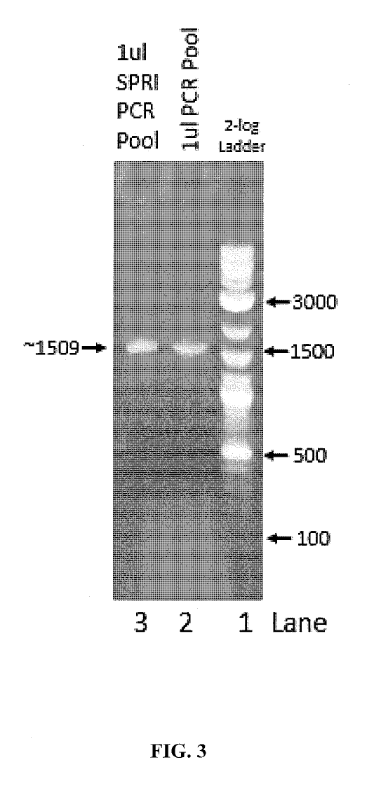

3. Samples were analyzed on a 0.8% agarose gel to check purity. The gel result is shown in FIG. 3. The crude pool sample is in lane 2, SPRI purified sample is in lane 1 and lane 3 contains 2-log ladder DNA standard (New England Biolabs, #N3200).

Proteinase K Protocol:

Long term storage of purified DNA requires proteinase K treatment to inactivate purification enzymes, which will act to degrade DNA over time. If DNA is to be kept more than 48 hours, please use the rapid treatment protocol below:

Using 800units/ml proteinase K from New England Biolabs (#P8107), add 1 microliter Proteinase K per 10 microliters phi29 template reaction (0.092 units/microliter), incubate at 40° C. for 30 minutes, incubate at 95° C. for 10 minutes to inactivate Proteinase K, store at 4° C.

Additional Reagents and Materials:

1.5 milliliter tubes (96) or 96 well 0.5 milliliter sample block (1) for resuspending sample. 1.5 milliliter microfuge tubes are available from multiple commercial suppliers (for example, Dot Scientific Inc., #RN1700-GST). 96 well Assay Block 500 microliters are available from multiple suppliers (for example, Dot Scientific Inc., #PC93241-NS9).

Sterile inoculating loops (96) are available from multiple commercial suppliers (for example, BD Calibrated Disposable Inoculating Loops, Green, #220214 (25×10 pack, 250 total) or #220215 (20×50 pack, 1000 total)).

96 well plates (2; 0.2 milliliter) for sample lysis and purification. 96 well plates are available from multiple commercial suppliers (for example, Thermo Scientific, ThermoFast 96, Semi-Skirted, natural, #AB-0900).

Adhesive film (3) for covering 96-well plates is available from multiple commercial suppliers (for example, Thermo AB-0558).

SPRI beads (100 microliters) for purification of 1500 bp 16S rRNA PCR product, available from commercial suppliers (for example, Agencourt Ampure XP, #A63880, Beckman Coulter).

Sterile Laboratory Grade Water (˜3 milliliters) is available from multiple commercial suppliers (for example, Fisher Biotech Grade Water BP24854).

Suggested items for high throughput parallel processing:

Repeater Pipette with multi-volume tips, available from multiple commercial suppliers (for example, Eppendorf Repeater Stream).

Multichannel Pipette; 0.5-10 microliter working volume, available from multiple commercial suppliers (for example, Rainin Pipet-lite XLS, L-10).

Reagent Troughs useable with 8-channel for smaller volumes (for example, 25 milliliter ThermoScientific 809611 Reagent Reservoir with Divider part#14-387-072).

Aluminum 96 well plate working rack for incubating lysis mix, PCR mix on ice (for example, Stratagene cat# 410094, or LightLabs cat#A-7079).

96 well plate centrifuge (available through commercial suppliers)

Suggested items for QC of 96 samples:

0.8% agarose gel (available through commercial suppliers)

Gel loading dye (available through commercial suppliers)

2 kilobase (kb) DNA ladder or equivalent (available through commercial suppliers)

Suggested items for long term DNA storage:

Proteinase K, 800 units/ml, available from New England Biolabs (#P8107).

One or more embodiments of the present invention have been described. Nevertheless, it will be understood that various modifications may be made without departing from the spirit and scope of the invention. Accordingly, other embodiments are within the scope of the following claims.

What is claimed is:

1. A method for high throughput DNA purification and multiplex sample tracking, for simultaneous high throughput sequencing of a target DNA amplicon from many biological samples, comprising:
a. amplifying whole genome DNA from biological samples in a high throughput format, wherein the biological samples are selected from crude biological samples or partially-purified biological samples;
b. for each sample, amplifying one or more target DNA sequences from the amplified genomic DNA from step (a), wherein each targeted DNA sequence has a unique DNA barcode added during amplification that uniquely identifies the sample of origin;
c. conducting high throughput DNA sequencing on the pooled target DNA sequences from the pooled barcoded samples from step (b); and
d. sorting the DNA sequences obtained in step (c) according to the unique DNA barcode, and using the sorted DNA sequences to identify a microbe containing the target DNA sequence in each sample.

2. The method of claim 1, further comprising, prior to step (a), lysing cells in the biological sample so as to release DNA from the cells.

3. The method of claim 2, wherein the lysing is carried out using reagents selected from the group consisting of: enzymes such as lysozyme or proteinase, a base such as KOH or NaOH, a detergent such as nonyl phenoxypolyethoxylethanol (NP-40), 3-[(3-cholamidopropyl)dimethylammonio]-1-propanesulfonate (CHAPS); C₁₄H₂₂O(C₂H₄O)ₙ (n=9-10) (Triton X-100), or sodium dodecyl sulfate.

4. The method of claim 1, wherein the high throughput format is selected from the group consisting of: at least six samples, at least twenty-four samples, at least 48 samples, at least ninety-six samples, at least 384 samples or at least 1536 samples.

5. The method of claim 1, wherein the biological sample is selected from the group consisting of: feces, cell lysate, tissue, blood, tumor, tongue, tooth, buccal swab, phlegm, mucous, wound swab, skin swab, vaginal swab, or any other biological material or biological fluid originally obtained from a human, animal, plant, or environmental sample.

6. The method of claim 1, wherein the amplifying in step (a) uses a DNA polymerase capable of producing high yields of purified DNA from the biological sample.

7. The method of claim 6, wherein the polymerase is phi29 DNA polymerase.

8. The method of claim 1, wherein the amplifying in step (b) employs a polymerase chain reaction (PCR).

9. The method of claim 1, wherein the unique DNA tag in step (b) comprises a DNA sequence of two or more bases.

10. The method of claim 1, wherein the target DNA sequence is selected from a human, microbial, animal, plant or viral gene sequence.

11. The method of claim 1, wherein the target DNA sequence is selected from a 16S rRNA, 23S rRNA, eukaryotic 18S rRNA, human HLA, microbial toxin producing genes, microbial pathogenicity genes, microbial plasmid genes, human immune system genes, immune system components and other variable genetic regions of non-human organisms.

12. The method of claim 11, wherein the target DNA sequence is 16S rRNA.

13. The method of claim 1, wherein the high throughput DNA sequencing in step (c) is a next-generation sequencing (NGS) method.

14. The method of claim 13, wherein the next-generation sequencing method is selected from: single-molecule real-time sequencing, ion semiconductor sequencing, pyrosequencing, sequencing by synthesis, sequencing by ligation and chain termination sequencing.

15. The method of claim 1, wherein the sorting in step (d) employs computer implemented methods.

16. A kit for identifying microbes in a biological sample, comprising:
a. a DNA polymerase capable of producing high yields of purified DNA from a biological sample;
b. control samples and corresponding primers for amplifying target DNA sequences from the control samples; and
c. experimental primers for amplification of one or more microbe target DNA sequences from the biological sample.

17. The kit of claim 15, wherein the DNA polymerase is phi29.

18. The kit of claim 15, wherein the microbe target DNA sequence is a 16S rRNA gene sequence.

19. The kit of claim 15, wherein the experimental primers comprise unique DNA barcodes corresponding to the biological sample.

20. The kit of claim 15, wherein the kit further comprises one or more reagents to lyse cells selected from the group consisting of: enzymes such as lysozyme or proteinase, a base such as KOH or NaOH, a detergent such as nonyl phenoxypolyethoxylethanol (NP-40), 3-[(3-cholamidopropyl)dimethylammonio]-1-propanesulfonate (CHAPS); C₁₄H₂₂O(C₂H₄O)ₙ (n=9-10) (Triton X-100), or sodium dodecyl sulfate.
